(19) United States
(12) Patent Application Publication
(10) Pub. No.: US 2006/0052807 A1
(43) Pub. Date: Mar. 9, 2006

(54) Anti-contamination scalpel

(76) Inventor: I-Chi Lee, Taipei (TW)

Correspondence Address: BIRCH STEWART KOLASCH & BIRCH, PO BOX 747, FALLS CHURCH, VA, 22040-0747, US

(21) Appl. No.: 10/934,373

(22) Filed: Sep. 7, 2004

(51) Int. Cl. A61B 17/32

(52) U.S. Cl. 606/167

(57) ABSTRACT

An anti-contamination scalpel accomplishes by following processes: a. sand blasting on the surface of the scalpels made of raw stainless steel for the first time; b. sand blasting on the surface of the scalpels with finer material for the second time; c. surface wash with acid and high temperature burning to wash away impurities, dust and oil stain; d. detailed cleaning and dry-up processes; e. spray anti-contamination coating on the surface of the scalpels, then places the scalpels into oven for high temperature baking to form a protective layer. The protective layer can reduce contamination on the surface of the scalpels to achieve better duration and practicability.

### BACKGROUND OF THE INVENTION

I. Field of the Invention

This invention relates generally to a scalpel and, more specifically, to an anti-contamination scalpel that has the metal surface with anti-contamination function, especially to apply the high temperature baking method combines the surface process of the scalpel of electrical surgical operations.

II. Description of the Prior Art

Heretofore, it is known that scalpels of the electrical surgical operations do not have to be very sharp but must have conductivity and anti-contamination character, such that to avoid bad conductivity due to contact patients' wound during operation, that will effect the process of operation. Most of the known scalpels of electrical surgical operation have a Teflon layer for above purpose; however, after a long period of time, the Teflon layer falls off or becomes thin, the conductivity of scalpels wears out or reduces, contamination still happens.

The known high temperature burning method can melt small metal or non-metal particles to change the format or structure of these particles, in the fields of steel making, metallurgy, non-iron metal metallurgy, metal refining, papermaking and mechanical components applied in other industrial fields, the metal particles can be shaped into different format and still maintain their original physical property, such as stability in high temperature, high stress, heat and electrical conductivity, chemical stability and wear resisting. The high temperature burning method applied on scalpels can achieve different mechanical, heat stability and electrical conductivity requirements by adjustment metal and non-metal particle composition.

### SUMMARY OF THE INVENTION

It is therefore a primary object of the invention to provide an anti-contamination scalpel that offers surface process method on the surface of the scalpels for better duration and practicability.

In order to achieve the objective set forth, an anti-contamination scalpel in accordance with the present invention accomplishes by following processes: a. sand blasting on the surface of the scalpels made of raw stainless steel for the first time; b. sand blasting on the surface of the scalpels with finer material for the second time; c. surface wash with acid and high temperature burning to wash away impurities, dust and oil stain; d. detailed cleaning and dry-up processes; e. spray anti-contamination coating on the surface of the scalpels, then places the scalpels into oven for high temperature baking to form a protective layer.

Based on the processes described above, the scalpels of electrical surgical operations are processed on the scalpel itself, the blade or edge; during electrical surgical operations, the high frequency electrical power can reduce the burned black tissues while cutting, also achieve styptic effect. The surface process method only forms protective layer on the surface, the physical character of the material are not altered, only a hard metal thin layer on the surface, the scalpels not only maintain good conductivity, after a long period of time the layer will not fall off to achieve better duration and practicability.

### BRIEF DESCRIPTION OF THE DRAWINGS

The accomplishment of the above-mentioned object of the present invention will become apparent from the following description and its accompanying drawings which disclose illustrative an embodiment of the present invention, and are as follows:

FIG. 1 is a flow chart of the present invention;

FIG. 2 is a time vs. temperature chart of the present invention.

### DESCRIPTION OF THE PREFERRED EMBODIMENT

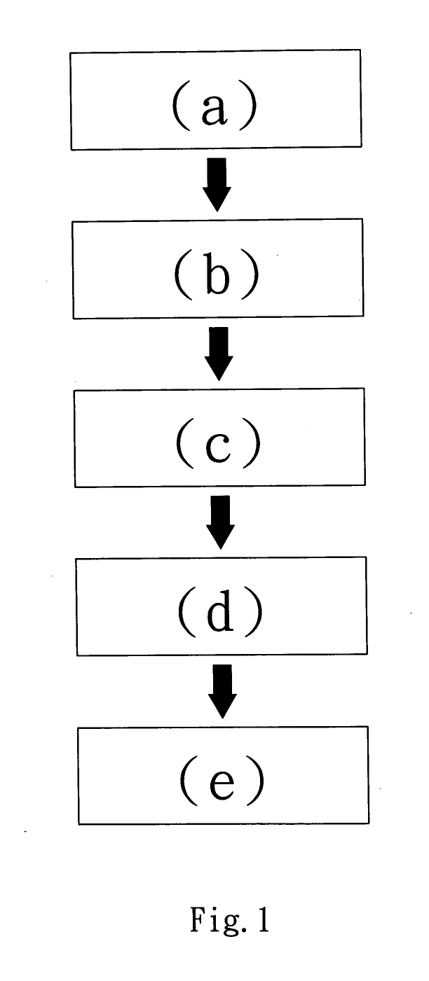

Referring to FIG. 1, the present invention processes through following procedures, the procedures are described as following:
a. Sand blasting on the surface of the scalpels made of raw stainless steel for the first time, the sand blasting material is round bead in 100#, and the round bead can be metal, ceramic or glass.
b. Sand blasting on the surface of the scalpels with finer material for the second time, the sand blasting material is round bead in 200#, and the round bead can be metal, ceramic or glass.
c. Surface wash with acid and high temperature burning to wash away impurities, dust and oil stain.
d. Detailed cleaning and dry-up processes.
e. Spray anti-contamination coating on the surface of the scalpels, then place the scalpels into oven for high temperature baking to form a protective layer, the thickness of the protective layer is about 0.02 mm.

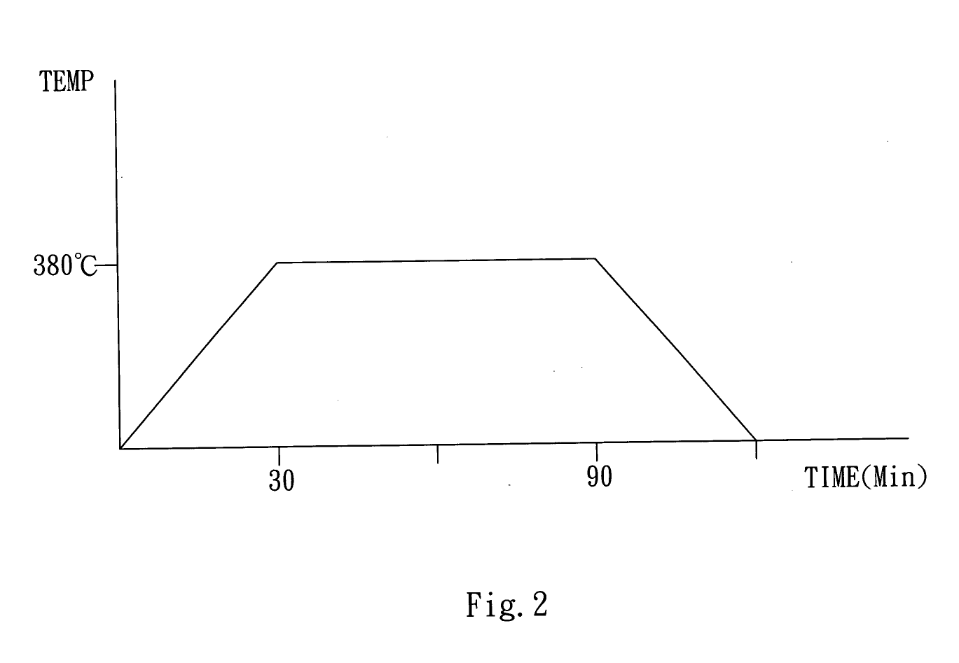

FIG. 2 shows the time vs. temperature chart of the high temperature baking, the temperature is about 380° C., the duration is about 120 minutes, the coating material can be arranged by manufacturers to use non-toxic materials, such as Xylan 1820/PQ1941 Dark Grey #673.

While a preferred embodiment of the invention has been shown and described in detail, it will be readily understood and appreciated that numerous omissions, changes and additions may be made without departing from the spirit and scope of the invention.

What is claimed is:

1. An anti-contamination scalpel accomplishes by following processes:
a. Sand blasting on the surface of the scalpels made of raw stainless steel for the first time;
b. Sand blasting on the surface of the scalpels with finer material for the second time;
c. Surface wash with acid and high temperature burning to wash away impurities, dust and oil stain;
d. Detailed cleaning and dry-up processes;
e. Spray anti-contamination coating on the surface of the scalpels, then places the scalpels into oven for high temperature baking to form a protective layer.

2. The anti-contamination scalpel recited in claim 1, wherein the sand blasting material is round bead. Can be metal, ceramic or glass.

3. The anti-contamination scalpel recited in claim 2, wherein said round bead is metal bead.

4. The anti-contamination scalpel recited in claim 2, wherein said round bead is ceramic bead.

5. The anti-contamination scalpel recited in claim 2, wherein said round bead is glass bead.

6. The anti-contamination scalpel recited in claim 1, wherein the sand blasting material in said process a. is round bead in 100#.

7. The anti-contamination scalpel recited in claim 1, wherein the sand blasting material in said process b. is round bead in 200#.

8. The anti-contamination scalpel recited in claim 1, wherein the thickness of the protective layer is about 0.02 mm.

9. The anti-contamination scalpel recited in claim 1, wherein in said high temperature baking process, the temperature is about 380° C., the duration is about 120 minutes.

10. The anti-contamination scalpel recited in claim 1, wherein said coating material can be Xylan 1820/PQ1941 Dark Grey #673.
